(19) United States
(12) Patent Application Publication
(10) Pub. No.: US 2026/0034198 A1
(43) Pub. Date: Feb. 5, 2026

(54) NANOZYME-LOADED NUCLEUS PULPOSUS MATRIX HYDROGEL MICROSPHERE

(71) Applicant: Union Hospital Tongji Medical College Huazhong University of Science and Technology, Wuhan (CN)

(72) Inventors: Yizhong PENG, Wuhan (CN); Xuanzuo Chen, Wuhan (CN); Zengwu Shao, Wuhan (CN); Ying Bai, Guangzhou City (CN); Zilong Rao, Guangzhou City (CN); Wei Wu, Wuhan (CN); Kanglu Li, Wuhan (CN); Xiangcheng Qing, Wuhan (CN); Hui Lin, Wuhan (CN); Yihan Yu, Wuhan (CN)

(21) Appl. No.: 19/357,427

(22) Filed: Oct. 14, 2025

Related U.S. Application Data
(63) Continuation of application No. PCT/CN2023/093021, filed on May 9, 2023.

(30) Foreign Application Priority Data
Apr. 26, 2023 (CN) 202310458530.X

(51) Int. Cl. A61K 38/44; A61K 9/06; A61K 9/16; A61K 33/08

(52) U.S. Cl. CPC A61K 38/443; A61K 9/06; A61K 9/1617; A61K 9/1623; A61K 33/08

(57) ABSTRACT

A nanozyme-loaded nucleus pulposus matrix hydrogel microsphere is provided. A simple-to-prepare LOX—MnO₂ nanozyme is provided, and the simple-to-prepare LOX—MnO₂ nanozyme is loaded onto the GDNP, and an LOX—MnO₂-loaded and glucose-enriched decellularized nucleus pulposus hydrogel microsphere, namely the nanozyme-loaded nucleus pulposus matrix hydrogel microsphere is formed through a new two-stage temperature-controlling microfluidic system.

### CROSS-REFERENCE TO RELATED APPLICATIONS

This application is a continuation of International Patent Application No. PCT/CN2023/093021, filed May 9, 2023, and claims priority of Chinese Patent Application No. 202310458530.X, filed on Apr. 26, 2023. The entire contents of International Patent Application No. PCT/CN2023/093021 and Chinese Patent Application No. 202310458530.X are incorporated herein by reference.

### TECHNICAL FIELD

The present disclosure belongs the field of biomaterials technology, and particularly relates to a nanozyme-loaded nucleus pulposus matrix hydrogel microsphere.

### BACKGROUND

Intervertebral disc degeneration (IDD) is a common degenerative disease that may lead to intervertebral disc (IVD) herniation, reduced spinal mobility, and the induction of radiculopathy and chronic low back pain (LBP). LBP may significantly impact patients' quality of life and increase social and economic burdens. However, due to insufficient understanding of the disease's pathogenesis and the mechanisms underlying postoperative adverse reactions, commonly used methods such as nucleotomy, vertebral fusion surgery, local blockades, and conservative drug treatments have not yielded satisfactory results. Developing new therapeutic strategies for IDD is an urgent task for clinical researchers. Stem cell-based tissue engineering and biomaterial technologies are fundamental pillars of regenerative medicine. Marrow mesenchymal stem cells (MSCs) possess characteristics such as proliferative capacity, multi-lineage differentiation potential, ease of isolation, and low immunogenicity, making MSCs highly promising for IDD treatment. Currently, MSCs have been applied in numerous clinical trials targeting IDD and have been proven to be safe and well-tolerated.

Due to the avascular nature of normal IVD, nutrients are supplied by surrounding blood vessels and transported to the center of the IVD through diffusion. As the nutrient concentration decreases with increasing distance from the blood vessels, the IVD is relatively nutrient-deficient and may only support a limited number of cells. When IDD occurs, factors such as marrow space occlusion, cartilage end-plate calcification, and lumbar artery atherosclerosis further reduce nutrient supply. In stem cell therapy, the implantation of exogenous MSCs leads to a sharp increase in cell numbers within the IVD. The implanted MSCs compete with the remaining native IVD cells for nutrients, exacerbating the nutrient deficiency in the IVD and further causing cell death. Although MSCs may effectively regulate inflammation and stimulate extracellular matrix production, MSCs must survive to exert their functions. Therefore, providing sufficient energy, particularly glucose, is essential to maintain the activity of implanted MSCs. The imbalance between nutrient demand and supply after MSC implantation, especially in the context of IDD, reduces the efficiency of stem cell therapy.

The hypoxic microenvironment in the IVD dictates that anaerobic glycolysis is the primary energy metabolism pathway for nucleus pulposus cells (NPCs). This pathway leads to the accumulation of glycolytic metabolites within the intervertebral disc. It has been reported that lactate, the primary metabolite of anaerobic glycolysis, is present in higher concentrations in intervertebral disc tissues of IDD compared to blood and other tissues. High concentrations of lactate may impair the activity of MSCs and IVD cells. Therefore, when using stem cells for treatment, it is also necessary to consider eliminating the adverse effects of metabolites to improve the energy metabolism environment and maintain cell survival and function.

Lactate oxidase (LOX) catalyzes the oxidation of lactate to produce pyruvate and hydrogen peroxide (H₂O₂), where H₂O₂ is a key component of reactive oxygen species that may induce oxidative stress and lead to cell death in IDD. Shen et al. designed a manganese dioxide (MnO₂)-based nanozyme system. Using MnO₂ to catalyze the decomposition of H₂O₂ into oxygen and water, this system may metabolize the H₂O₂ generated by lactate consumption and provide additional O˜2˜ to promote further lactate consumption. However, the synthesis of the MnO₂-based nanozyme in this report is relatively complex, requiring four steps and an additional heat source to reach a reaction temperature of 60 degrees Celsius (° C.), which is not conducive to large-scale industrial production and clinical application. Therefore, there is a need to improve the synthesis method of nanozymes. Moreover, nanozymes face challenges such as small size, easy diffusion, and rapid cellular clearance, making it difficult to achieve long-term local enrichment. Therefore, there is an urgent need for a suitable delivery system to maintain their local efficacy.

### SUMMARY

To address the issues in the prior art mentioned above, the present disclosure proposes a nucleus pulposus matrix hydrogel microsphere loaded with nanozymes.

To achieve the above objective, the present disclosure provides the following technical solutions.

A nanozyme-loaded nucleus pulposus matrix hydrogel microsphere, where lactate oxidase and manganese dioxide nanozyme are simultaneously loaded, and a glucose content in the nucleus pulposus matrix hydrogel microsphere is greater than or equal to 2.5-5 millimolar (mM).

A preparation method of the nanozyme-loaded nucleus pulposus matrix hydrogel microsphere, including: adding lactate oxidase into MnO₂ nanodispersion, and obtaining lactate oxidase-manganese dioxide nanozyme (LOX—MnO₂) nanozyme particles after dispersion and centrifugation; adding genipin into a decellularized nucleus pulposus tissue digestive solution, and obtaining a pre-gel solution by adjusting pH and an osmotic pressure; and adding the genipin, a glucose solution and the LOX—MnO₂ nanozyme particles into the pre-gel solution again, pumping into a microfluidic chip to shear into droplets, and then gelling to obtain the nanozyme-loaded nucleus pulposus matrix hydrogel microsphere.

In an embodiment, the preparation method of the nanozyme-loaded nucleus pulposus matrix hydrogel microsphere, specifically including following steps:
S1, synthesis of LOX—MnO₂ nanozyme particles
dissolving potassium permanganate in double distilled water, then dropping a poly(allylamine hydrochloride) (PAH) solution, stirring at 25° C. for 30 minutes (min), putting a mixture containing MnO₂ nanoparticles into a dialysis bag for dialysis, and freezing-drying to obtain freeze-dried powder; adding the freeze-dried powder into the double distilled water to obtain the MnO₂ nanodispersion, adding the lactate oxidase, stirring at 25° C. and 200 revolutions per minute (rpm) for 30 min, and finally centrifuging at 20000 rpm for 30 min, and washing to obtain the LOX—MnO₂ nanozyme particles;
S2, the nanozyme-loaded nucleus pulposus matrix hydrogel microsphere
adding pepsin containing hydrochloric acid (HCl) with a concentration of 1% weight/volume (w/v) into decellularized nucleus pulposus tissue powder after a pretreatment for a digestion treatment, centrifuging an obtained digestive solution, adding the genipin, adjusting the pH to 7.3-7.5, and adjusting the osmotic pressure to an isotonic state to form the pre-gel solution, and denoting as a decellularized nucleus pulposus (DNP) pre-gel solution; and
adding glucose and the genipin into the DNP pre-gel solution to obtain a glucose-enriched decellularized nucleus pulposus (GDNP) pre-gel solution, then continuously adding the LOX—MnO₂ nanozyme particles in the S1 into the GDNP pre-gel solution, pumping an obtained LOX—MnO₂-loaded and glucose-enriched decellularized nucleus pulposus (LMGDNP) pre-gel solution into the microfluidic chip, shearing to obtain the droplets, and gelling the droplets to obtain an LOX—MnO₂-loaded and glucose-enriched decellularized nucleus pulposus hydrogel microsphere, namely the nanozyme-loaded nucleus pulposus matrix hydrogel microsphere.

In an embodiment, in the S2, the pretreatment refers to: repeatedly freeze-thawing fresh nucleus pulposus blocks in liquid nitrogen and a 37° C. water bath 5 times, then treating with 2 weight percent (wt %) Triton X-100 under room temperature shaking conditions for 48 hours (h); and centrifuging and discarding a supernatant, treating with 1 wt % sodium dodecyl sulfate (SDS) under the room temperature shaking conditions for 24 h, and finally treating with 200 Units per milliliter (U/mL) DNase under 37° C. shaking conditions for 12 h to form decellularized nucleus pulposus tissue blocks, washing with sterile water, and pulverizing to obtain decellularized nucleus pulposus tissue powder.

In an embodiment, in the S2, the digestion treatment refers to digesting at 25° C. for 48 h.

In an embodiment, in the S2, a mass ratio of the GDNP to the LOX—MnO₂ nanozyme particles is 100:1.

The present disclosure also provides a two-stage temperature-controlling microfluidic system, where the system is used for preparing the nanozyme-loaded nucleus pulposus matrix hydrogel microsphere, and specifically consisting of a polydimethylsiloxane chip, a microinjection pump, a heating magnetic stirrer, polytetrafluoroethylene tubes and a silicone tube.

The present disclosure also provides an application of the nanozyme-loaded nucleus pulposus matrix hydrogel microsphere.

Compared with the prior art, the present disclosure has the following advantages and technical effects.

The present disclosure utilizes microfluidic technology to prepare GDNP hydrogel microspheres, with a glucose content of up to 2.5-5 mM in the prepared hydrogel microspheres, providing nutritional supply and differentiation cues for MSCs. In addition, the present disclosure also constructs a simple-to-prepare LOX—MnO₂ nanozyme particle, which is loaded onto GDNP to form the LMGDNP hydrogel microsphere, so as to remove excess lactate in the intervertebral disc and reduce the damage of metabolic by-products to cells. The system is simple in preparation, convenient in storage, injectable, easy for mass production and has potential for clinical application. The system aims to regulate the nutritional imbalance and harsh microenvironment after stem cell implantation in the intervertebral disc, thereby improving the efficiency of stem cell therapy and providing a new strategy for the treatment of IDD.

### BRIEF DESCRIPTION OF THE DRAWINGS

The drawings, which form a part of the present disclosure, are provided to further illustrate the present disclosure. The illustrative embodiments and descriptions in the present disclosure are intended to explain the present disclosure and do not constitute any limitation to the present disclosure. In the drawings:

FIG. 1 is a schematic diagram illustrating construction of LOX—MnO₂ nanozyme particles (LM).

FIG. 2 is a schematic diagram illustrating construction of LMGDNP hydrogel microspheres.

FIG. 3 shows a relationship between glucose concentration and cell viability.

FIG. 4 shows a two-stage temperature-controlling microfluidic system (TSTC-MS).

FIG. 5A shows transmission electron microscope (TEM) images of PAH-coated MnO₂ nanoparticles and LM.

FIG. 5B shows size distribution of nanoparticles.

FIG. 5C shows size distribution of MnO₂ nanoparticles and LOX—MnO₂ nanozyme, with data presented as mean±standard deviation, n=3 (indicating three repeated experiments).

FIG. 6A shows the effect of LOX loading on the ζ potential of PAH-MnO₂ nanoparticles.

FIG. 6B shows scanning electron microscope (SEM) images and elemental analysis of nanoparticles.

FIG. 6C shows the evaluation of protein distribution of LOX, MnO₂, and LM nanoparticles using sodium dodecyl sulfate-polyacrylamide gel electrophoresis (SDS-PAGE) silver staining.

FIG. 6D shows Ultraviolet-visible spectroscopy (UV-Vis) spectra of LOX, MnO₂, and LM nanoparticles.

FIG. 7A shows the evaluation of decellularization efficiency using Hematoxylin and Eosin (HE) staining, scale bar: 100 micrometers (μm).

FIG. 7B shows the evaluation of decellularization efficiency using 4′,6-diamidino-2-phenylindole (DAPI) staining, scale bar: 100 μm.

FIG. 7C shows quantitative analysis of DNA content in fresh nucleus pulposus tissue blocks (FNP-B) and decellularized nucleus pulposus tissue blocks (DNP-B).

FIG. 7D shows the glycosaminoglycan (GAG) analysis of FNP-B and DNP-B to evaluate the content of glycosaminoglycans in the tissue. The data is presented as mean±standard deviation, n=3, *p<0.05, **p<0.01.

FIG. 8A shows the collection of microspheres dispersed in liquid paraffin.

FIG. 8B shows size distribution and microstructure of microspheres, with scale bars of 100 μm and 2.5 μm.

FIG. 9A shows an accumulated release curve of LOX from LMGDNP based on bicinchoninic acid (BCA) assay.

FIG. 9B shows lactate clearance efficiency of LOX—MnO₂ nanozyme and microspheres.

FIG. 10 shows the efficiency evaluation of LOX—MnO₂ nanozyme and microspheres in promoting H₂O₂ decomposition.

FIG. 11 shows Calcein-acetoxymethyl ester/propidium iodide (Calcein-AM/PI) fluorescence images of bone marrow mesenchymal stem cells (BMSCs) loaded on different microspheres or cultured as cell spheroids for 14 days, scale bar: 100 μm.

FIG. 12A shows the cell viability of BMSCs loaded on different microspheres or cultured as cell spheroids, detected by the cell counting kit-8 (CCK8) assay.

FIG. 12B shows the cell viability of BMSCs cultured for 24 hours in microsphere feeding systems with or without lactate, detected and evaluated by the CCK8 assay.

FIG. 12C shows the survival of BMSCs cultured for 24 hours in microsphere feeding systems with or without lactate, evaluated by the Calcein-AM/PI fluorescence staining, scale bar: 100 μm.

FIG. 13A shows the expression of markers (Krt19, CD24, Col2, and Acan) of NPCs in BMSCs cultured on microspheres for 21 days using immunofluorescence, with scale bar: 100 μm.

FIG. 13B shows relative mRNA expression of Krt19, CD24, Col2, and Acan in BMSCs cultured on microspheres for 14 and 21 days, with data presented as mean±SD, n=3, ns: not significant, *p<0.05, **p<0.01, ***p<0.001.

### DETAILED DESCRIPTION OF THE EMBODIMENTS

Various exemplary embodiments of the present disclosure are described in detail below. This detailed description should not be construed as limiting the present disclosure but rather as providing a more detailed explanation of certain aspects, features, and embodiments of the present disclosure.

It should be understood that the terminology used herein is for the purpose of describing particular embodiments only and is not intended to limit the present disclosure. Additionally, any numerical range recited herein is intended to include every intermediate value between the upper and lower limits of the range. Any stated value or range of values includes each smaller range within the stated range, as well as any other stated or intermediate value within the stated range. The upper and lower limits of these smaller ranges may be independently included or excluded from the range.

Unless otherwise specified, all technical and scientific terms used herein have the same meaning as commonly understood by one of ordinary skill in the art to which the present disclosure belongs. Although only preferred methods and materials are described herein, any methods and materials similar or equivalent to those described may be used in the practice or testing of the present disclosure. All publications mentioned herein are incorporated by reference to disclose and describe the methods and/or materials related to the publications. In the event of any conflict with the incorporated literature, the content of this specification shall prevail.

Various modifications and changes may be made to the specific embodiments described in this specification without departing from the scope or spirit of the present disclosure, as will be apparent to those skilled in the art. Other embodiments derived from the description of the present disclosure will be apparent to those skilled in the art. The specification and embodiments herein are exemplary only.

The terms “comprising,” “including,” “having,” “containing,” and the like, as used herein, are open-ended terms meaning including but not limited to.

As used herein, “room temperature” refers to 25±2° C. unless otherwise specified.

All raw materials used in the following examples of the present disclosure are commercially available.

In recent years, decellularized extracellular matrix (dECM) has attracted increasing attention in regenerative medicine due to its excellent biocompatibility, biomimetic fiber structure, and bioactivity. In previous studies, a dECM hydrogel derived from porcine sciatic nerve was prepared, and microspheres were fabricated using a microfluidic system. These microspheres demonstrated good cell compatibility and sustained release of small molecule drugs. Additionally, the dECM hydrogel derived from nucleus pulposus (NP) matrix effectively promoted tissue-specific differentiation of stem cells into NPCs. Therefore, the present disclosure aims to prepare NP matrix-based hydrogel microspheres as a delivery system for stem cells and nanozymes.

The present disclosure utilizes microfluidic technology to prepare GDNP hydrogel microspheres to provide nutritional support and differentiation cues for MSCs. In addition, according to the present disclosure, a simple-to-prepare LOX—MnO₂ nanozyme is constructed and loaded onto the GDNP to form LMGDNP hydrogel microspheres (LMGDNP pre-gel solution), so as to remove excess lactate in the intervertebral disc and reduce cellular damage caused by metabolic by-products. The system is simple in preparation, convenient in storage, injectable, easy for mass production and has potential for clinical application. The system aims to regulate the nutritional imbalance and harsh microenvironment after stem cell implantation in the intervertebral disc, thereby improving the efficiency of stem cell therapy and providing a new strategy for the treatment of IDD.

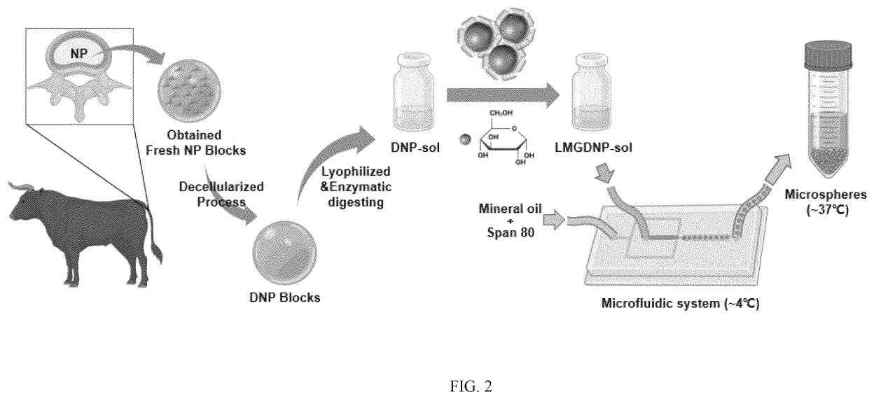

The main preparation process is as follows: the NP tissue collected from a cattle tail is decellularized to remove cellular components, and a pre-gel solution, namely DNP pre-gel solution, is formed through pepsin-mediated enzymatic digestion. By adding genipin, LOX—MnO₂, and glucose to the DNP pre-gel solution, the LMGDNP pre-gel solution is obtained, and then pumped into a microfluidic chip to form LMGDNP droplets through shear force upon encountering an oil phase. The droplets are gelled by immersion in a 37° C. water bath within a silicone tube, and LMGDNP microspheres are collected (with reference to FIG. 2).

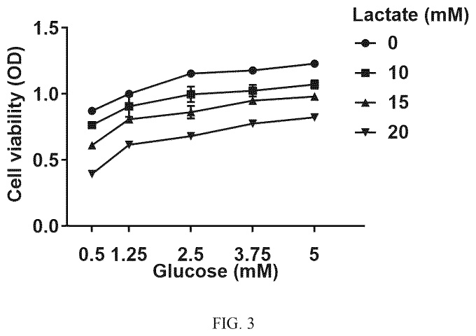

When NPCs are treated with lactate (0, 10, 15, 20 mM) and glucose (0.5, 1.25, 2.5, 3.75, 5 mM), cell viability gradually decreases with increasing lactate concentration. After the glucose concentration reaches 2.5 mM, cell viability remains at a high level (with reference to FIG. 3). Therefore, the glucose concentration in the microspheres in subsequent experiments is chosen to be no less than 2.5 mM, with 5 mM being preferred in the following embodiments of the present disclosure.

### Embodiment 1

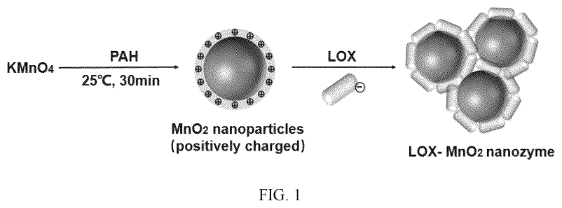

(1) Preparation of LOX—MnO₂ Nanozyme Particles (Schematic Diagram of LOX—MnO₂ Nanoenzyme Construction as Shown in FIG. 1)

64 milligrams (mg) of KMnO₄ are dissolved in 18 milliliter (mL) of double distilled water. Then, 2 mL of PAH solution with a concentration of 36 milligrams per milliliter (mg/mL) is added dropwise to the solution. After reacting at 25° C. for 30 minutes, the obtained mixture containing MnO₂ nanoparticles is placed in a dialysis bag (molecular weight 3 kiloDalton (kDa)) and dialyzed in double distilled water for one week, and then freeze-dried to obtain freeze-dried powder for yield calculation. To prepare the LOX—MnO₂ nanoparticles, the freeze-dried powder is added to double distilled water to obtain 3.6 mg/mL MnO₂ nanodispersion. 50 Units (U) of LOX is added to 1 mL of the MnO₂ nanodispersion, and the mixture is stirred at 25° C. (200 rpm) for 30 minutes, then centrifuged at 20000 rpm for 30 minutes and washed three times with PBS to obtain LOX—MnO₂ nanoparticles (denoted to as LM nanoparticles).

### (2) Preparation of LMGDNP Hydrogel Microspheres (LMGDNP)

Step 1, within 6 hours after slaughter, the NP tissue is obtained from the tail vertebrae of cattle and cut into small pieces, referred to as FNP-B. The FNP-B is subjected to five cycles of freezing in liquid nitrogen and thawing in a 37° C. water bath, treated with 2 wt % TritonX-100 for 48 h under shaking condition, centrifuged to discard the supernatant, treated with 1 wt % SDS for 24 h under shaking conditions, and treated with 200 U/mL DNA enzyme at 37° C. for 12 h to remove the residual DNA, forming DNP-B. The DNP-B is washed with sterile water, ground into powder, and digested in pepsin (0.1% weight by volume (w/v)) containing 1% w/v HCl (0.01 mole per liter (M)) at 25° C. for 48 hours to obtain a digestive solution. The digestive solution is centrifuged to remove undissolved particles. The pH of the digestive solution is adjusted to 7.4 using 0.1 M NaOH, and genipin (0.02% w/v) is added, and the osmotic pressure is adjusted to the isotonic state to form a pre-gel solution. Glucose solution and genipin (0.02% w/v) are added to the DNP pre-gel solution to obtain a GDNP pre-gel solution with a glucose concentration of 5 mM. LM nanoparticles and genipin (0.02% w/v) are added to the GDNP pre-gel solution (mass ratio of LM nanoparticles to GDNP pre-gel solution is 1:100) to obtain the LMGDNP pre-gel solution, which is stored at 4° C. until use.

Step 2, AutoCAD software is used to design, and the silicon wafer mold is manufactured by photolithography. Polydimethylsiloxane (PDMS) base is mixed with a curing agent (mass ratio of PDMS to curing agent is 10:1, curing agent item number: Sylgard 184) and degassed for 15 minutes, then poured onto the silicon wafer mold and degassed for 2 hours, and then cured at 70° C. for 4 hours, and then the PDMS is peeled off from the silicon wafer mold. Two 1.0 millimeter (mm) diameter holes for the inlet channels and one 2.0 mm diameter hole for the outlet channel are drilled into the PDMS to obtain the chip. A flat PDMS cover plate is also prepared on the silicon wafer. Finally, the PDMS surface is activated using an oxygen plasma surface treatment instrument, and the PDMS cover plate is carefully placed onto the chip and left at 70° C. for 30 minutes to obtain a microfluidic chip.

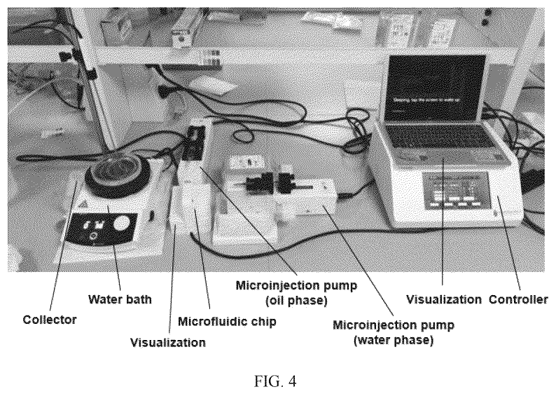

Step 3, the microspheres are prepared using a novel two-stage temperature-controlling microfluidic system (TSTC-MS) (as shown in FIG. 4). The TSTC-MS consists of a PDMS chip, a microinjection pump with two channels, a heatable magnetic stirrer, two polytetrafluoroethylene tubes (inner diameter=1.0 mm), and a silicone tube (inner diameter=2.0 mm). The aqueous phase (LMGDNP pre-gel solution) and oil phase (liquid paraffin containing 20% volume/volume (v/v) Span 80) are mixed in the microfluidic chip at 4° C., and an emulsion of water-in-oil droplets is formed by shear force. The formation of droplets is observed using a small inverted laboratory microscope. The flow rate ratio of the aqueous to oil phase is 1:10. The emulsion flows into a silicone tube immersed in a 37° C. water bath for gelation to form microspheres. The microspheres are then dropped into a two-layer liquid phase consisting of ether and phosphate buffered saline (PBS) solution to remove the mineral oil. The microspheres are concentrated in the PBS solution, centrifuged, and washed three times with PBS. The microspheres are stored at 37° C. for further use. For sterilization, the microspheres are centrifuged at 2000 rpm for 5 minutes to remove the PBS, resuspended in 75% ethanol at 25° C. for 24 hours, and finally washed three times with sterile PBS to remove residual ethanol, and LMGDNP hydrogel microspheres (LMGDNP) are obtained.

In the above S2, the aqueous phase LMGDNP pre-gel solution is replaced with DNP pre-gel solution and GDNP pre-gel solution, respectively, while keeping other microsphere preparation conditions unchanged, to obtain DNP microspheres and GDNP microspheres. These are used as controls for subsequent performance testing.

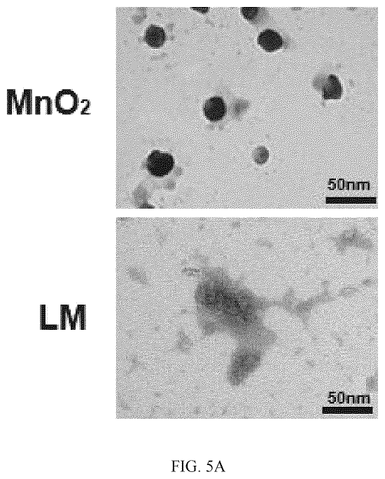

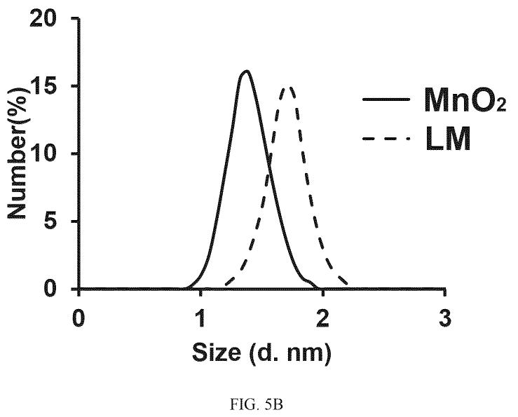

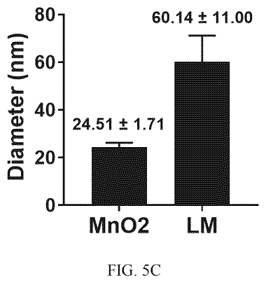

FIG. 5A-FIG. 5C show the morphology and size of the LOX—MnO₂ nanozyme. From FIG. 5A, it may be seen that the PAH-coated MnO₂ nanoparticles appear as stable colloidal dispersion, while the LOX loading alters the morphological structure. Meanwhile, the diameter of the LM nanozyme particles significantly increases after LOX loading (FIG. 5B and FIG. 5C).

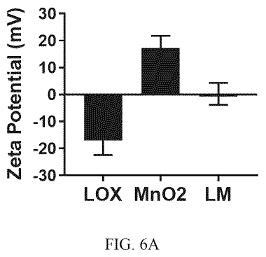

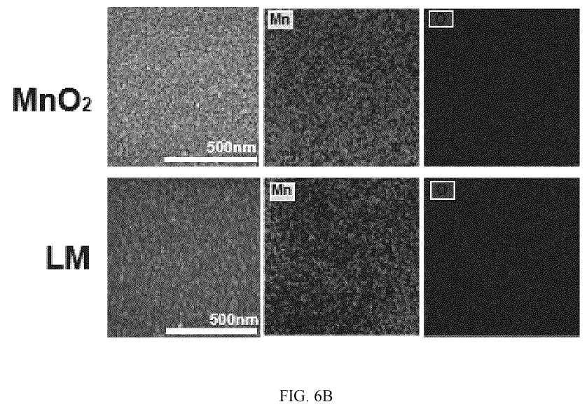

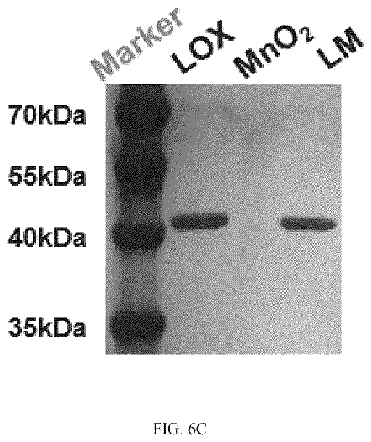

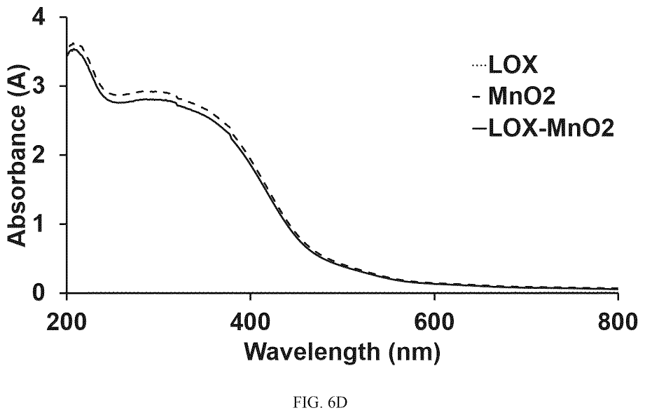

FIG. 6A-FIG. 6D show the composition and characteristics of the LOX—MnO₂ nanozyme. After LOX loading, the ζ potential of the MnO₂ nanoparticles decreases from approximately 20 millivolt (mV) to about 0 mV, forming a stable non-covalent complex (FIG. 6A). BCA assay determines the LOX loading capacity of the MnO₂ nanoparticles to be 54.18±6.97%. Energy dispersive spectrometry (EDS) is used to verify the elemental distribution, showing that manganese (Mn) and oxygen (O) are uniformly distributed in both LM and MnO₂ nanoparticles (FIG. 6B). LOX, MnO₂ and LM nanoparticles are delivered to SDS-PAGE and developed by silver staining. LOX and LM groups show similar bands at 40 kDa, indicating successful LOX loading on the MnO₂ nanoparticles (FIG. 6C). UV-Vis spectra show that the spectra of MnO₂ and LM are similar, indicating that LOX loading hardly changes the ultraviolet absorption properties of MnO₂ nanoparticles (FIG. 6D).

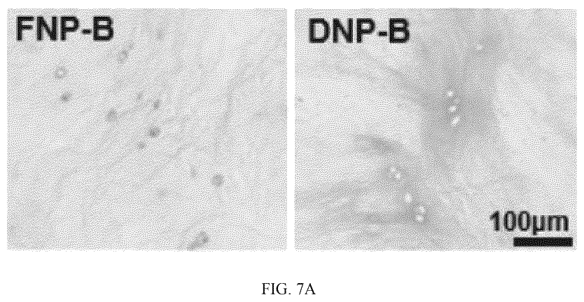

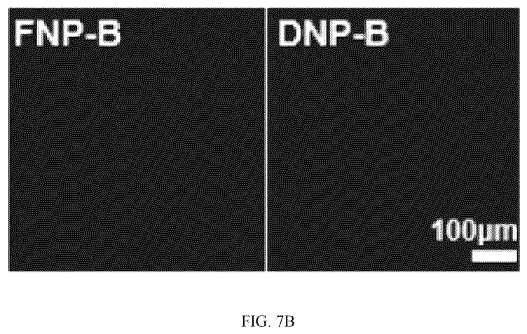

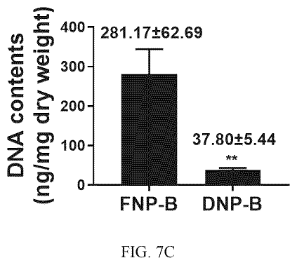

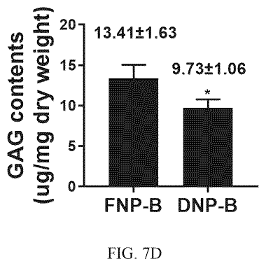

FIG. 7A-FIG. 7D show the efficacy evaluation of the decellularization treatment. HE staining show that cell components are removed after decellularization treatment, while extracellular matrix (ECM) remains during decellularization process (FIG. 7A). Similarly, DAPI staining show that cell nuclei are removed from the tissue, indicating that decellularization treatment is effective (FIG. 7B). DNA content decreases from 281.17±62.69 nanogram per milligram (ng/mg) in FNP-B to 37.80±5.44 ng/mg in DNP-B (FIG. 7C), while GAG (a major component of the extracellular matrix) decreases from 13.41±1.63 microgram per milligram (g/mg) in FNP-B to 9.73±1.06 g/mg in DNP-B (FIG. 7D), indicating that the decellularization process significantly removes cellular components while retaining most of the extracellular matrix.

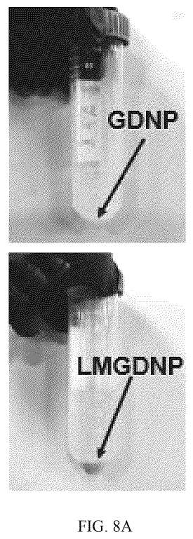

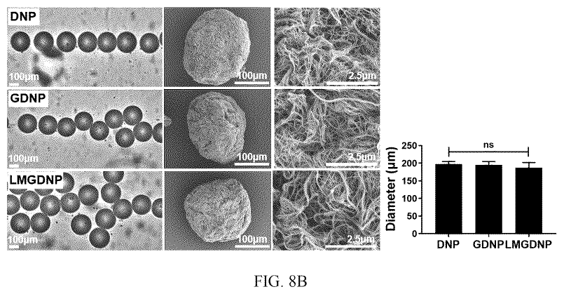

FIG. 8A-FIG. 8B show the macroscopic and microscopic morphology of the microspheres. The obtained microspheres are immersed in the oil phase and precipitate at the bottom (FIG. 8A). Under real-time observation, the droplet diameter is consistent (DNP: 197.63±7.34 μm; GDNP: 195.49±9.10 μm; LMGDNP: 187.94±14.36 μm), and the nanofiber structure of the collected microspheres is similar to the self-assembled nanostructure of DNP hydrogel (FIG. 8B), which may simulate the extracellular matrix microenvironment of nucleus pulposus to support cell attachment, proliferation, and directed differentiation.

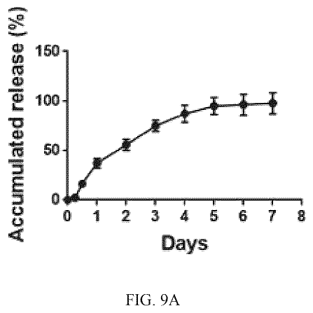

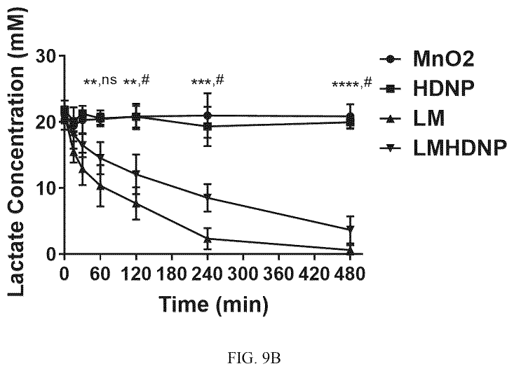

FIG. 9A-FIG. 9B show the release of LOX from LMGDNP over time. As shown by the BCA assay, LMGDNP exhibits a sustained release profile of LOX (FIG. 9A). Lactate concentration measurements at different time points show that lactate levels gradually decrease in both LMGDNP and LM groups, but LMGDNP appears less effective than LM, possibly due to the delayed release of LOX from LMGDNP into the enzymatic reaction system (FIG. 9B).

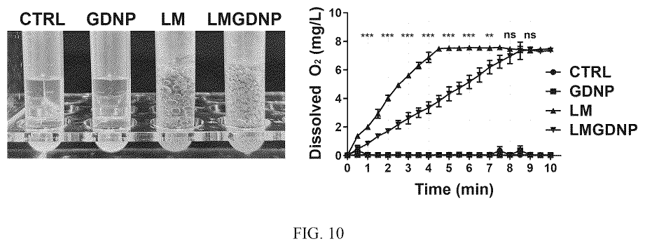

FIG. 10 shows the efficiency evaluation of LOX—MnO₂ nanozyme and microspheres in promoting H₂O₂ decomposition. LOX-mediated lactate clearance leads to the accumulation of H₂O₂, causing oxidative stress. By evaluating the clearance efficiency of H₂O₂ through the detection of oxygen production in the system, it is found that LMGDNP and LM significantly promote oxygen production, while the delayed oxygen production in the LMGDNP group indicates that LM is released slowly in the system, resulting in a lower concentration of MnO₂ nanoparticles compared to the LM group. Both LMGDNP and LM exhibit catalase-like activity in eliminating H₂O₂ and producing oxygen to improve the hypoxic microenvironment in the IVD.

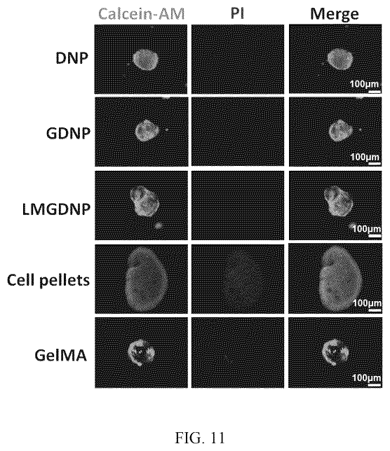

FIG. 11 shows the Calcein-AM/PI fluorescence images of BMSCs loaded on DNP, GDNP, LMGDNP, and Gelatin Methacryloyl (GelMA, purchased from Suzhou Yongqinquan Intelligent Equipment Co., Ltd.) (BMSCs are obtained by co culturing with microspheres on low adhesion pore plates for 24 h) microspheres or cultured as cell spheroids for 14 days. Calcein-AM/PI staining shows that BMSCs cultured on GelMA, DNP, GDNP, and LMGDNP does not show significant cell death on day 14, while propidium iodide-positive (PI-positive) staining significantly increases in the center of cell spheroids, indicating significant cell death in the center of the spheroids. Compared to cell spheroids, cells cultured on the surface of microspheres may have better access to nutrients and oxygen, leading to higher cell survival rates.

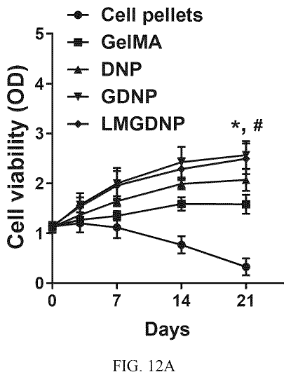

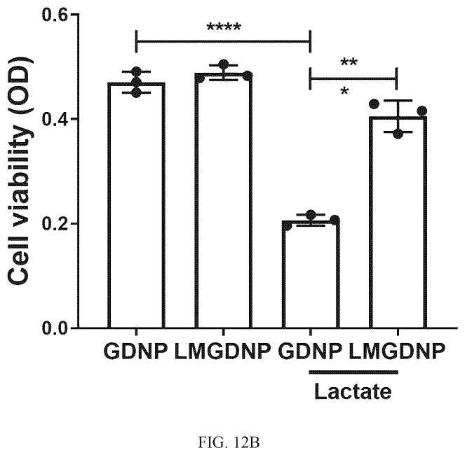

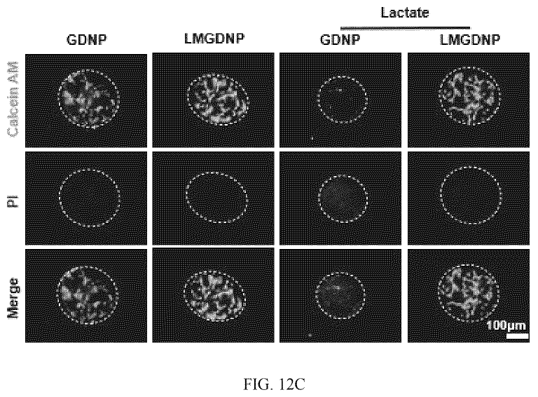

FIG. 12A-FIG. 12C shows the biocompatibility of the microspheres. CCK8 assay shows that cell viability increases more significantly in the DNP group compared to GelMA and cell spheroids (FIG. 12A), indicating that dECM microspheres are more suitable for cell survival than GelMA. Additionally, GDNP and LMGDNP groups show better cell viability than the DNP group (FIG. 12A), which may be related to the ability of glucose to enhance cell viability (FIG. 12B). The lactate concentration in the nucleus pulposus of patients with LBP ranges from 2 to 6 mM. To evaluate the ability of the microspheres to consume lactate, lactate (6 mM) is added to the microsphere culture system. CCK-8 assay shows that cell viability significantly decreases in the lactate+GDNP group compared to the lactate+LMGDNP group (FIG. 12C).

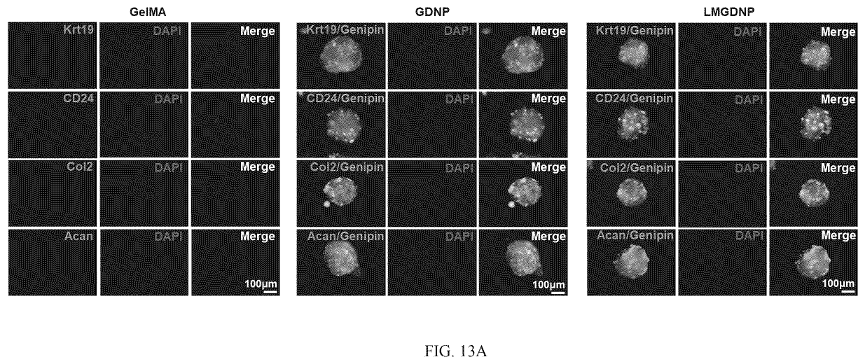

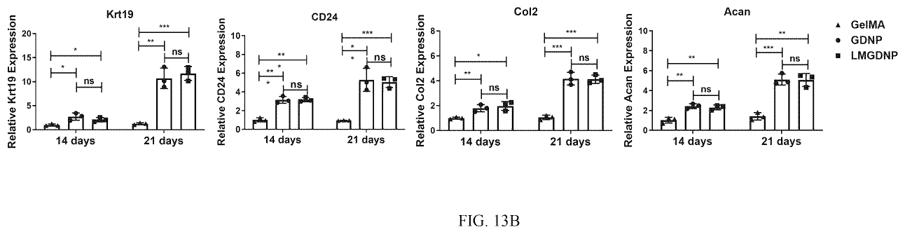

To evaluate the differentiation-promoting ability of dECM microspheres, BMSCs are cultured on GelMA, GDNP, and LMGDNP microspheres for 14 and 21 days, and the cell surface markers (Krt19, CD24, Col2 and Acan) of NPCs are detected. On the 21st day, immunofluorescence staining of Krt19, CD24, Col2, and Acan shows significant positive staining in the GDNP and LMGDNP groups, while the above indicators are not significantly positive in the GelMA group (FIG. 13A). Polymerase chain reaction (PCR) analysis shows that the mRNA expression of Krt19, CD24, Col2, and Acan is significantly increased in the GDNP and LMGDNP groups (FIG. 13B).

The above description is only a preferred embodiment of the present disclosure and does not limit the scope of the present disclosure. Any modifications or substitutions that may be easily conceived by one of ordinary skill in the art within the technical scope disclosed in the present disclosure should be covered by the protection scope of the present disclosure. Therefore, the protection scope of the present disclosure should be defined by the claims.

What is claimed is:

1. A nanozyme-loaded nucleus pulposus matrix hydrogel microsphere, wherein lactate oxidase and manganese dioxide nanozyme are simultaneously loaded, and a glucose content in the nanozyme-loaded nucleus pulposus matrix hydrogel microsphere is greater than or equal to 2.5-5 mM.

2. A preparation method of the nanozyme-loaded nucleus pulposus matrix hydrogel microsphere according to claim 1, comprising: adding the lactate oxidase into MnO₂ nanodispersion, and obtaining LOX—MnO₂ nanozyme particles after dispersion and centrifugation;
adding genipin into a decellularized nucleus pulposus tissue digestive solution, and obtaining a pre-gel solution by adjusting pH and an osmotic pressure; and adding the genipin, a glucose solution and the LOX—MnO₂ nanozyme particles into the pre-gel solution again, pumping into a microfluidic chip to shear into droplets, and then gelling to obtain the nanozyme-loaded nucleus pulposus matrix hydrogel microsphere.

3. The preparation method of the nanozyme-loaded nucleus pulposus matrix hydrogel microsphere according to claim 2, specifically comprising following steps:
S1, synthesis of LOX—MnO₂ nanozyme dissolving potassium permanganate in double distilled water, then dropping a poly(allylamine hydrochloride) solution, stirring at 25° C. for 30 min, putting a mixture containing MnO₂ nanoparticles into a dialysis bag for dialysis, and freezing-drying to obtain freeze-dried powder; and adding the freeze-dried powder into the double distilled water to obtain the MnO₂ nanodispersion, adding the lactate oxidase, stirring at 25° C. and 200 rpm for 30 min, and finally centrifuging at 20000 rpm for 30 min, and washing to obtain the LOX—MnO₂ nanozyme particles;
S2, the nanozyme-loaded nucleus pulposus matrix hydrogel microsphere adding pepsin containing HCl with a concentration of 1% w/v into decellularized nucleus pulposus tissue powder after a pretreatment for a digestion treatment, centrifuging an obtained digestive solution, adding the genipin, adjusting the pH to 7.3-7.5, and adjusting the osmotic pressure to an isotonic state to form the pre-gel solution, and denoting as a DNP pre-gel solution; and
adding glucose and the genipin into the DNP pre-gel solution to obtain a GDNP pre-gel solution, then continuously adding the LOX—MnO₂ nanozyme particles in the S1 into the GDNP pre-gel solution, pumping an obtained LMGDNP pre-gel solution into the microfluidic chip, shearing to obtain the droplets, and gelling the droplets to obtain an LOX—MnO₂-loaded and glucose-enriched decellularized nucleus pulposus hydrogel microsphere, namely the nanozyme-loaded nucleus pulposus matrix hydrogel microsphere.

4. The preparation method of the nanozyme-loaded nucleus pulposus matrix hydrogel microsphere according to claim 3, wherein in the S2, the pretreatment refers to: repeatedly freeze-thawing fresh nucleus pulposus blocks in liquid nitrogen and a 37° C. water bath 5 times, then treating with 2 wt % polyethylene glycol octylphenyl ether under room temperature shaking conditions for 48 h; and centrifuging and discarding a supernatant, treating with 1 wt % sodium dodecyl sulfate (SDS) under the room temperature shaking conditions for 24 h, and finally treating with 200 U/mL DNase under 37° C. shaking conditions for 12 h to form decellularized nucleus pulposus tissue blocks, washing with sterile water, and pulverizing to obtain the decellularized nucleus pulposus tissue powder.

5. The preparation method of the nanozyme-loaded nucleus pulposus matrix hydrogel microsphere according to claim 3, wherein in the S2, the digestion treatment refers to digesting at 25° C. for 48 h.

6. The preparation method of the nanozyme-loaded nucleus pulposus matrix hydrogel microsphere according to claim 3, wherein in the S2, a mass ratio of the GDNP pre-gel solution to the LOX—MnO₂ nanozyme particles is 100:1.

7. A two-stage temperature-controlling microfluidic system, wherein the two-stage temperature-controlling microfluidic system is used for preparing the nanozyme-loaded nucleus pulposus matrix hydrogel microsphere according to claim 1, and specifically consisting of a polydimethylsiloxane chip, a microinjection pump, a heating magnetic stirrer, polytetrafluoroethylene tubes and a silicone tube.
